(19) United States
(12) Patent Application Publication
(10) Pub. No.: US 2024/0210311 A1
(43) Pub. Date: Jun. 27, 2024

(54) METHOD AND SYSTEM FOR MEASURING GAS CONCENTRATION BY IDENTIFYING FEATURES OF UNKNOWN GAS

(71) Applicant: NANJING ANRONX ELECTRONICS TECHNOLOGY CO., LTD., Jiangsu (CN)

(72) Inventors: Shaosheng XU, Jiangsu (CN); Xiaojie DING, Jiangsu (CN); Hui WANG, Jiangsu (CN); Jia WANG, Jiangsu (CN); Yun QIU, Jiangsu (CN); Chuan ZHANG, Jiangsu (CN)

(73) Assignee: NANJING ANRONX ELECTRONICS TECHNOLOGY CO., LTD., Jiangsu (CN)

(21) Appl. No.: 18/518,569

(22) Filed: Nov. 23, 2023

Related U.S. Application Data
(63) Continuation of application No. PCT/CN23/78327, filed on Feb. 27, 2023.

(30) Foreign Application Priority Data
Dec. 22, 2022 (CN) 202211656520.9

(51) Int. Cl. G01N 21/31; G06F 30/27

(52) U.S. Cl. CPC G01N 21/31; G06F 30/27

(57) ABSTRACT

The present application discloses a method and a system for measuring a concentration of a gas to be measured by identifying features of an unknown gas. According to the method, a function model is constructed according to features of a known gas and features of a gas mixture, the features of the unknown gas are finally acquired by learning parameters in a training function, and the features of the unknown gas are incorporated into a feature library of the known gas, to provide more accurate parameters for obtaining the concentration of each component in the gas mixture through inversion calculation. The system includes a light source, a measuring cell, a spectrometer, a storage module, a learning and training module, an initialization module, and an inversion module.

### CROSS-REFERENCE TO RELATED APPLICATION

This application is a continuation of international PCT application serial no. PCT/CN2023/078327, filed on Feb. 27, 2023, which claims priority benefit of China patent application No. 202211656520.9 filed on Dec. 22, 2022. The entirety of each of the above-mentioned patent applications is hereby incorporated by reference herein and made a part of this specification.

### TECHNICAL FIELD

The present application relates to the technical field of gas concentration detection, and particularly relates to a method and a system for measuring a concentration of a gas to be measured by identifying features of an unknown gas.

### BACKGROUND

Spectral detection technology is widely used for quality monitoring of water, air and soil in China, particularly for detection of concentrations of industrial pollution gases. This spectral detection method is simple but convenient, and achieves more accurate detection. However, in practical applications, in addition to known pollution gases that need to be measured, many unknown gases are also contained in gases emitted by industries, which causes absorption spectra overlapping and serious cross interference, thus greatly affecting the accuracy of measurement.

In order to solve the above problems, in the prior art, a filtering method or a mathematical algorithm is mainly used to eliminate the interference of an unknown gas on the measurement accuracy. According to the filtration method, before entry into a measuring cell, a gas mixture is treated first by using a filter (such as a hydrocarbon remover) to filter out some gases that do not need to be detected by means of chemical reactions. However, only specific known gases can be filtered through this method, and remaining unknown gases cannot be identified yet. The commonly used mathematical algorithms include compensation methods and correction methods, such as a least square method, a regression method, a support vector machine, etc., where the least square method and the regression method are not robust and have poor inversion accuracy, and are easily affected by singular points, while the support vector machine raises relatively high requirements for samples. These algorithms are only effective for specific gases in specific scenes, and cannot completely eliminate the impact of an unknown interference gas on measurement precision.

### SUMMARY

The present application provides a method and a system for measuring a concentration of a gas to be measured by identifying features of an unknown gas, which can eliminate the impact of an unknown interference gas on gas measurement precision to the greatest extent, and greatly improve the accuracy and precision of a gas measurement system.

The method for measuring a concentration of a gas to be measured by identifying features of an unknown gas includes the following steps:
S1, injecting standard samples of m known gases into a measuring cell of a gas analyzer, scanning in a wide spectral range to obtain an absorption spectrum of each sample, further obtaining a feature Dᵢ of each sample gas, and establishing a feature library D of the known gases;
S2, injecting a gas mixture into the measurement cell of the gas analyzer, and scanning in a wide spectral range to obtain a feature d of the gas mixture, where the gas mixture includes m known gases and n unknown gases, and features of the unknown gases are defined as Duⱼ;
S3, defining and initializing a known gas weight parameter w and an unknown gas weight parameter wu, and constructing a squared loss function and an objective function according to the known gas feature Dᵢ, the known gas weight parameter w, the unknown gas feature Duⱼ, the unknown gas weight parameter wu, and the gas mixture feature d;
S4, learning and training each parameter in the squared loss function until the value of the objective function is less than the set value or the number of learning times reaches the target number, and obtaining the final unknown gas feature Duⱼ; and
S5, adding the final unknown gas feature Duⱼ to the known gas feature library D, and obtaining the concentration of a gas to be measured in the gas mixture through gas concentration inversion calculation.

The squared loss function described in S3 is defined as follows:

$$θ = {( {\sum }_{i = 1}^{m} {w}_{i} * {D}_{i} + {\sum }_{j = 1}^{n} w {u}_{j} * D {u}_{j} - d )}^{2}$$

and the objective function min(θ) is constructed according to the squared loss function.

The unknown gas feature Duⱼ in S2 is defined as follows:

A gas feature function base Φ and a gas feature function space are established, the unknown gas parameter Puⱼ is defined and initialized, and the unknown gas feature Duⱼ is constructed according to the gas feature function base Φ and the unknown gas parameter Puⱼ.

An expression of constructing the unknown gas feature Duⱼ according to the gas feature function base Φ and the unknown gas parameter Puⱼ is as follows:

$$D {u}_{j} = P {u}_{j} * Φ$$

The process of learning and training each parameter in the squared loss function until the value of the objective function is less than the set value or the number of learning times reaches a target number, and obtaining the final unknown gas feature Duⱼ described in S4, includes the following steps:
S41, using an iterative algorithm to learn and train the weight parameters w and wu and the unknown gas feature parameter Puⱼ, and recording an iteration number K;
S42, determining whether the remainder of K divided by a given integer N is zero, performing S43 if yes, and performing S44 if not;
S43, constructing a function of a correlation Rⱼ between the unknown gas feature Duⱼ and the known gas feature library D, initializing a correlation coefficient r, and determining whether Rⱼ is greater than r; if yes, re-initializing the unknown gas feature parameter Puⱼ, and performing S41; if not, performing S44; and
S44, determining whether the iteration number K reaches a set maximum K_max or whether the value of the objective function min(θ) is less than a set value ε; when at least any one of the above conditions is determined to be “yes”, stopping the iteration and calculating the final unknown gas feature Duⱼ; and when any of the above conditions is determined to be “not”, performing S41 and continuing learning and training.

Preferably, in S41, the iterative algorithm is a gradient projection method with momentum.

In S43, the function of the correlation Rⱼ between the unknown gas feature Duⱼ and the known gas feature library D is as follows:

$$r e s = ( D {u}_{j} * D ) * {( D * D )}^{- 1} * D - D {u}_{j} ,$$

$${R}_{j} = \frac{1 - res * res}{D {u}_{j} * D {u}_{j}} .$$

In S5, the final unknown gas feature Duⱼ is added to the known gas feature library D, and the concentration of the gas to be measured in the gas mixture is obtained by inversion calculation according to the least square method.

A system for measuring a concentration of a gas to be measured by identifying features of an unknown gas, adopts the above method for measuring a concentration of a gas to be measured by identifying features of an unknown gas, which includes a light source, a measurement cell, and a spectrometer, where the light source is sequentially connected to the measurement cell and the spectrometer, and further includes a storage module, a learning and training module, an initialization module, and an inversion module; the gas to be measured is injected into the measurement cell, and light emitted by the light source passes through the measurement cell filled with the gas to be measured, is received by the spectrometer and converted into a digital signal and then inputted to the storage module and the learning and training module respectively; the storage module is configured to store feature data of a variety of known gases; the learning and training module is configured to construct a squared loss function model between unknown gas features and known gas features and perform learning and training; the initialization module is connected to the learning and training module, and is configured to initialize the parameter data in the function model; the final unknown gas feature data obtained by learning and training is inputted into the storage module; the inversion module is connected with the storage module and configured to calculate the concentration of the gas to be measured, and the known gas feature data in the storage module and the final unknown gas feature data acquired by calculation are inputted into the inversion module for inversion calculation.

### BRIEF DESCRIPTION OF THE DRAWINGS

FIG. 1 is a flow chart of steps of a method for measuring a concentration of a gas to be measured by identifying features of an unknown gas provided by the present application;

FIG. 2 is a schematic flow diagram of an Embodiment 1 of the present application;

FIG. 3 is a chart of comparison between an unknown gas feature curve obtained by applying the method in the Embodiment 1 of the present application and an actual gas feature curve;

FIG. 4 is a comparison table of concentrations of a gas to be measured obtained in the Embodiment 1 of the present application;

FIG. 5 is a schematic flow diagram of an Embodiment 2 of the present application;

FIG. 6 is a chart of comparison between a feature curve of two unknown mixed gases obtained by applying the method in the Embodiment 2 of the present application and an actual gas feature curve;

FIG. 7 is a comparison table of concentrations of a gas to be measured obtained in the Embodiment 2 of the present application; and

FIG. 8 is a schematic diagram of a system for measuring a concentration of a gas to be measured by identifying features of an unknown gas provided by the present application.

### DETAILED DESCRIPTIONS OF THE EMBODIMENTS

In order to illustrate the present application more clearly, the present application will be further described below in combination with the accompanying drawings.

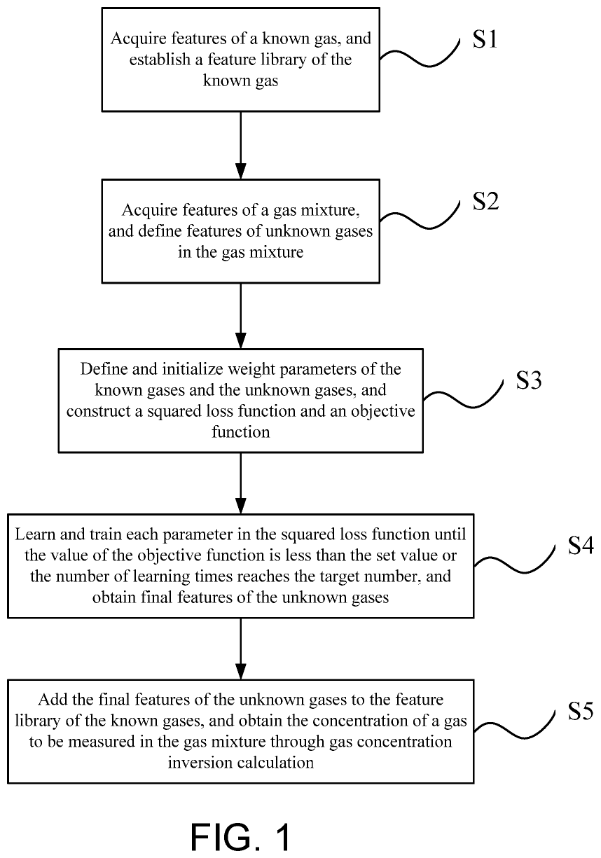

As shown in FIG. 1, a method for measuring a concentration of a gas to be measured by identifying features of an unknown gas, includes the following steps:
S1, standard samples of m known gases are injected into a measuring cell of a gas analyzer, scanning in a wide spectral range is performed to obtain an absorption spectrum of each sample, a feature Dᵢ of each sample gas is further obtained, and a feature library D of the known gases is established;
S2, a gas mixture is injected into the measurement cell of the gas analyzer, and scanning in a wide spectral range is performed to obtain the feature d of the gas mixture, where the gas mixture includes m known gases and n unknown gases, and features of the unknown gases are defined as Duⱼ;
S3, a known gas weight parameter w and an unknown gas weight parameter wu are defined and initialized, and a squared loss function is constructed according to the known gas feature Dᵢ, the known gas weight parameter w, the unknown gas feature Duⱼ, the unknown gas weight parameter wu, and the gas mixture feature d;
S4, the weight parameters w and wu are learned and trained until the minimum value of the squared loss function is less than a set value or the number of learning times reaches a target number, and the final unknown gas feature Duⱼ is obtained; and
S5, the final unknown gas feature Duⱼ is added to the known gas feature library D, and the concentration of each component gas in the gas mixture is obtained through gas concentration inversion calculation.

The method of the present application aims to obtain the features of the unknown gas in a gas mixture, constructs a function model according to features of a known gas and features of the gas mixture, and finally obtains features of the unknown gas through learning and training. Features of any unknown gas different from the known gas can be obtained through this method, without limitation by any scene or gas type. The features of the unknown gas are incorporated into a feature library of the known gas, to provide more accurate parameters for obtaining the concentration of each component in the gas mixture through inversion calculation. The present application can effectively reduce cross interference caused by spectrum overlapping, eliminate the impact of an unknown interference gas on gas measurement precision to the greatest extent, and greatly improve the accuracy and precision of a gas measurement system.

The squared loss function described in S3 is defined as follows:

$$θ = {( {\sum }_{i = 1}^{m} {w}_{i} * {D}_{i} + {\sum }_{j = 1}^{n} w {u}_{j} * D {u}_{j} - d )}^{2}$$

and the objective function min(θ) is constructed according to the squared loss function,
where m represents serial numbers of different known gases, and n represents serial numbers of different unknown gases.

The unknown gas feature Duⱼ in S2 is defined as follows:
a gas feature function base Φ and a gas feature function space are established, the unknown gas parameter Puⱼ is defined and initialized, and the unknown gas feature Duⱼ is constructed according to the gas feature function base Φ and the unknown gas parameter Puⱼ.

An expression of constructing the unknown gas feature Duⱼ according to the gas feature function base Φ and the unknown gas parameter Puⱼ is as follows:

$$D {u}_{j} = P {u}_{j} * Φ .$$

The process of learning and training each parameter in the squared loss function until the value of the objective function is less than the set value or the number of learning times reaches the target number, and obtaining the final unknown gas feature Duⱼ described in S4, includes the following steps:
S41, an iterative algorithm is used to learn and train the weight parameters w and wu and the unknown gas feature parameter Puⱼ, and the iteration number K is recorded;
S42, whether the remainder of K divided by a given integer N is zero is determined, S43 is performed if yes, and S44 is performed if not;
S43, a function of a correlation Rⱼ between the unknown gas feature Duⱼ and the known gas feature library D is constructed, a correlation coefficient r is initialized, and whether Rⱼ is greater than r is determined; if yes, the unknown gas feature parameter Puⱼ is re-initialized, and S41 is performed; if not, S44 is performed; and
S44, whether the iteration number K reaches the set maximum K_max or whether the value of the objective function min(θ) is less than the set value ε is determined; when at least any one of the above conditions is determined to be “yes”, the iteration is stopped and the final unknown gas feature Duⱼ is calculated and obtained; and when any of the above conditions is determined to be “not”, S41 is performed and learning and training are continued.

In S41, the iterative algorithm may be a gradient projection method with momentum, and of course any other gradient descent algorithm may also be used.

In S43, the function of the correlation Rⱼ between the unknown gas feature Duⱼ and the known gas feature library D is as follows:

$$res = ( D {u}_{j} * D ) * {( D * D )}^{- 1} * D - D {u}_{j} ,$$

$${R}_{j} = \frac{1 - res * res}{D {u}_{j} * D {u}_{j}} .$$

In S5, the final unknown gas feature Du is added to the known gas feature library D, and the concentration of the gas to be measured in the gas mixture is obtained by inversion calculation according to the least square method.

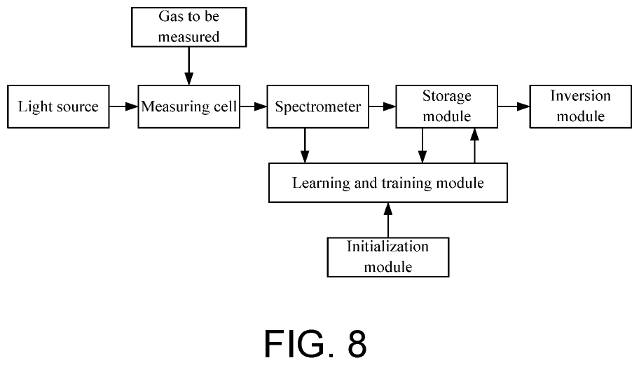

As shown in FIG. 8, a system for measuring a concentration of a gas to be measured by identifying features of an unknown gas, adopts the method for measuring a concentration of a gas to be measured by identifying features of an unknown gas in any one of the above embodiments, and includes a light source, a measuring cell, a spectrometer, a storage module, a learning and training module, an initialization module, and an inversion module, where the light source is sequentially connected to the measurement cell and the spectrometer, the gas to be measured is injected into the measurement cell, the light source enters the spectrometer through the gas in the measurement cell, and the spectrometer is configured to obtain the feature data of the gas to be measured and input such data into the storage module and the learning and training module respectively; the storage module is configured to store feature data of a variety of known gases; the learning and training module is configured to construct a squared loss function model between unknown gas features and known gas features and perform learning and optimal training; the initialization module is connected to the learning and training module, and is configured to initialize the parameter data in the function model; the learning and training module is configured to learn and train the parameter data in the function model; the final unknown gas feature data obtained by learning and training is inputted into the storage module; the inversion module is connected with the storage module, and the known gas feature data in the storage module and the final unknown gas feature data acquired by calculation are inputted into the inversion module for inversion calculation.

A non-transitory computer-readable storage medium, includes instructions for executing the method for measuring a concentration of a gas to be measured by identifying features of an unknown gas described in any one of the above embodiments.

An electronic device includes the non-transitory computer-readable storage medium; and one or more processors capable of executing the instructions of the non-transitory computer-readable storage medium.

### Embodiment 1

In order to make those skilled in the art better understand the present application, a more detailed embodiment is listed below for illustration. In this embodiment, a gas mixture composed of two known gases SO₂ and NO and one unknown gas is used as a test sample. The unknown gas used in this experiment is actually o-xylene, and a feature curve of the unknown gas obtained by use of this method is compared with a feature curve of the actual o-xylene to verify the method.

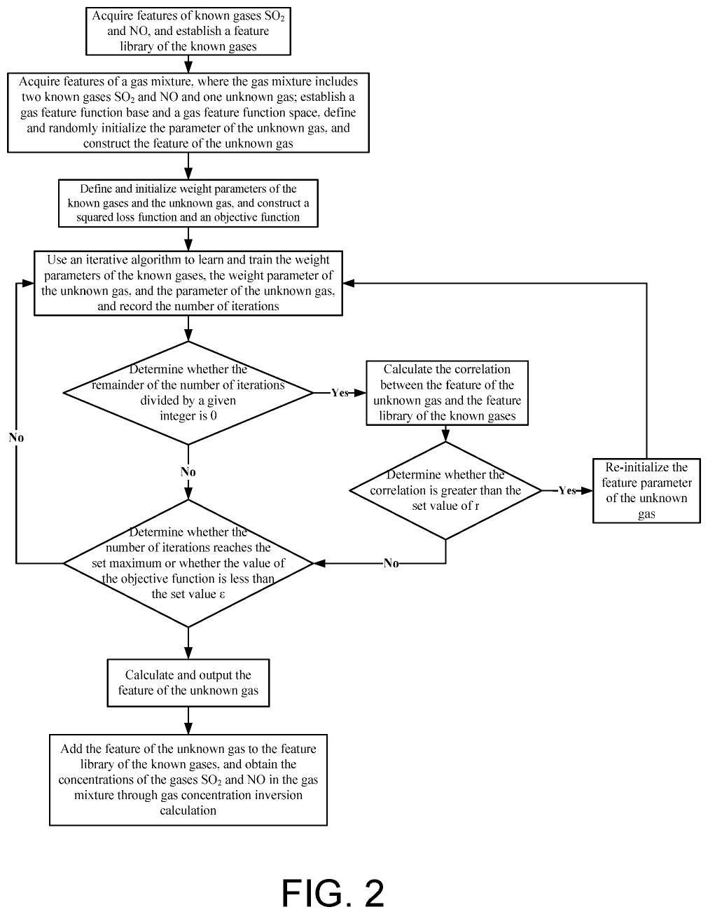

As shown in FIG. 2, a method for measuring a concentration of a gas to be measured by identifying features of an unknown gas includes the following steps:
S1, standard samples of the known gases SO₂ and NO are injected into a measuring cell of a gas analyzer respectively, scanning in a wide spectral range is performed to obtain an absorption spectrum of each sample, a feature D₁ of the SO₂ gas and a feature D₂ of the NO gas are further obtained, and a feature library D=[D₁, D₂] of the known gases is established;
S2, a gas mixture is injected into the measurement cell of the gas analyzer, and scanning in a wide spectral range is performed to obtain the feature d of the gas mixture, where the gas mixture includes two known gases SO₂ and NO and one unknown gas, and the feature of the unknown gas is defined as Du; a gas feature function base Φ and a gas feature function space are established, and the unknown gas parameter Puⱼ is defined and randomly initialized, where Puⱼ obeys the standard normal distribution; the feature of the unknown gas is constructed according to the gas feature function base Φ and the unknown gas parameter Puⱼ; Du=Puⱼ*Φ;
S3, weight parameters w₁ and w₂ of the gases SO₂ and NO and a weight parameter wu of the unknown gas are defined and initialized, where initial values of such parameters are all 0.01, and a squared loss function is constructed according to the known gas features D₁ and D₂, the known gas weight parameters w₁ and w₂, the unknown gas feature Du, the unknown gas weight parameter wu, and the gas mixture feature d as follows:

$$θ = {( {w}_{1} * {D}_{1} + {w}_{2} * {D}_{2} + w u * D u - d )}^{2}$$

and an objective function min(θ) is constructed according to the squared loss function;
S4, a gradient projection method with momentum is used to learn and train the weight parameters w₁ and w₂ of the gases SO₂ and NO, the unknown gas weight parameter wu, and the unknown gas parameter Puⱼ, an unknown gas feature Du is calculated, and the iteration number K is recorded;
S5, N is set as 100, whether the remainder of K divided by N is zero is determined, S6 is performed if yes, and S7 is performed if not;
S6, a correlation R between the unknown gas feature Du and the known gas feature library D is constructed as follows:

$$r e s = ( D u * D ) * {( D * D )}^{- 1} * D - D u , R = \frac{1 - res * res}{D u * D u} ,$$

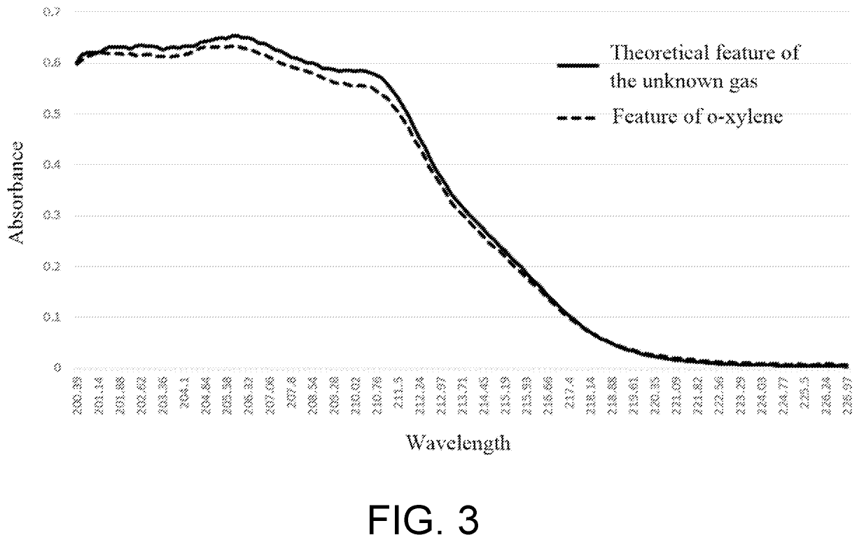

the correlation coefficient r is initialized as 0.3, and whether R is greater than 0.3 is determined; if yes, the unknown gas feature parameter Puⱼ is re-initialized, and S4 is performed; if not, S7 is performed;
S7, Kₘₐₓ=5000, ε=1e−3 is set, and when the iteration number K reaches 5000, the iteration is stopped and the final unknown gas feature D is calculated and obtained; a feature curve of the unknown gas obtained by use of this method is compared with a feature curve of the actual o-xylene, which is shown in FIGS. 3; and
S8, the unknown gas feature obtained by iterative calculation is added to the known gas feature library, and according to the Lambert-Beer's law: d=C_{SO2}*D₁+C_{NO}*D₂+Cᵤ*Du, the concentration of the gas to be measured in the gas mixture is obtained by inversion calculation according to the least square method. The specific formula is as follows:

$$( \begin{matrix}{c}_{So 2} \\ {c}_{N O} \\ {c}_{u}\end{matrix} ) = [ \begin{matrix}\begin{matrix}{D}_{1} * d & {D}_{2} * d\end{matrix} & Du * d\end{matrix} ] ( \begin{matrix}{D}_{1} * {D}_{1} & {D}_{2} * {D}_{1} & D u * {D}_{1} \\ {D}_{1} * {D}_{2} & {D}_{2} * {D}_{2} & D u * {D}_{2} \\ {D}_{1} * D u & {D}_{2} * D u & D u * D u\end{matrix} ) - 1.$$

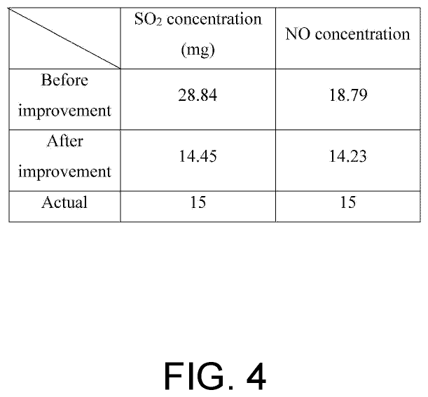

As shown in FIG. 4, in this embodiment, calculated final concentrations of the gases SO₂ and NO to be measured are 14.45% and 14.23% respectively; under the condition that the features of the unknown gas are unavailable, concentrations of the gases SO₂ and NO obtained by direct inversion calculation are 28.84% and 18.79%, respectively; and actual concentrations of the gases the gases and NO are 15% and 15%, respectively.

### Embodiment 2

In this embodiment, a gas mixture composed of two known gases SO₂ and NO and two unknown gases is used as a test sample. The unknown gases used in this experiment are actually NH₃ and CS₂, and a feature curve of the mixed unknown gases obtained by use of this method is compared with an actual feature curve of the gases NH₃ and CS₂ mixed to further verify the method.

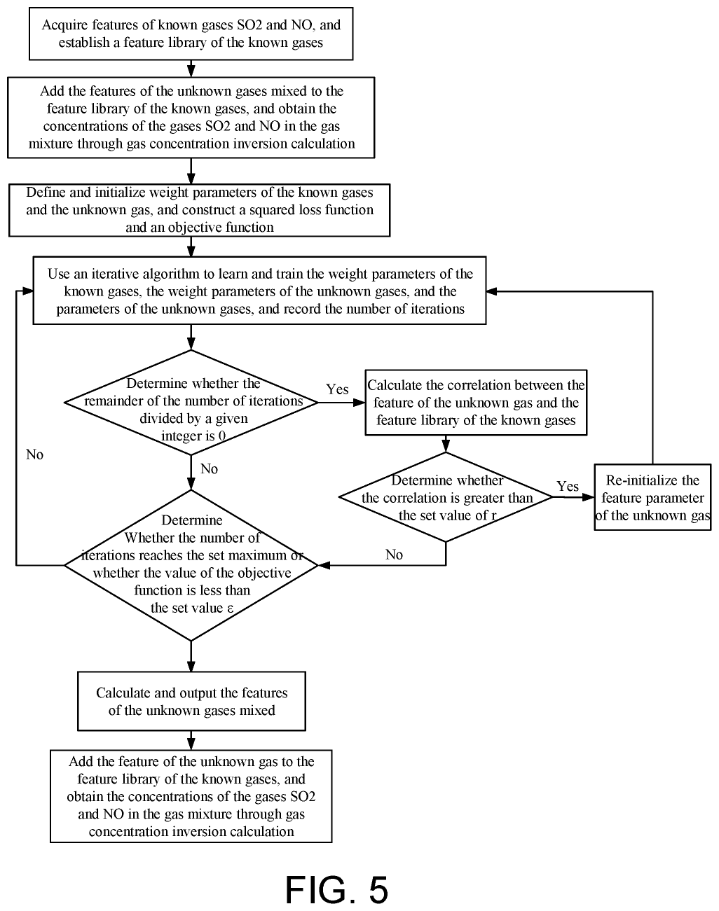

As shown in FIG. 5, the method for measuring a concentration of a gas to be measured by identifying features of an unknown gas includes the following steps:
S1, standard samples of the known gases SO₂ and NO are injected into a measuring cell of a gas analyzer respectively, scanning in a wide spectral range is performed to obtain an absorption spectrum of each sample, a feature D₁ of the SO₂ gas and a feature D₂ of the NO gas are further obtained, and a feature library D=[D₁, D₂] of the known gases is established;
S2, a gas mixture is injected into the measurement cell of the gas analyzer, and scanning in a wide spectral range is performed to obtain the feature d of the gas mixture, where the gas mixture includes two known gases SO₂ and NO and two unknown gases, and a feature of the unknown gas is defined as Du; a gas feature function base Φ and a gas feature function space are established, and an unknown gas parameter Puⱼ is defined and randomly initialized, where Puⱼ obeys the standard normal distribution; the feature of the unknown gas is constructed according to the gas feature function base Φ and the unknown gas parameter Puⱼ:Du=Puⱼ*Φ;
S3, weight parameters w₁ and w₂ of the gases SO₂ and NO and a weight parameter wu of the unknown gases are defined and initialized, where initial values of such parameters are all 0.01, and a squared loss function is constructed according to the known gas features D₁ and D₂, the known gas weight parameter w₁ and w₂, the unknown gas feature Du, the unknown gas weight parameter wu, and the gas mixture feature d as follows:

$$θ = {( {w}_{1} * {D}_{1} + {w}_{2} * {D}_{2} + w u * D u - d )}^{2}$$

and an objective function min(θ) is constructed according to the squared loss function;
S4, a gradient projection method with momentum is used to learn and train the weight parameters w₁ and w₂ of the gases SO₂ and NO, the weight parameter wu of the unknown gas, and the parameter Puⱼ of the unknown gas, a feature Du of the unknown gas is calculated, and the iteration number K is recorded;
S5, N is set as 100, whether the remainder of K divided by N is zero is determined, S6 is performed if yes, and S7 is performed if not;
S6, a correlation R between the unknown gas feature Du and the known gas feature library D is constructed as follows:

$$res = ( D u * D ) * {( D * D )}^{- 1} * D - D u , R = \frac{1 - res * res}{D u * D u} ,$$

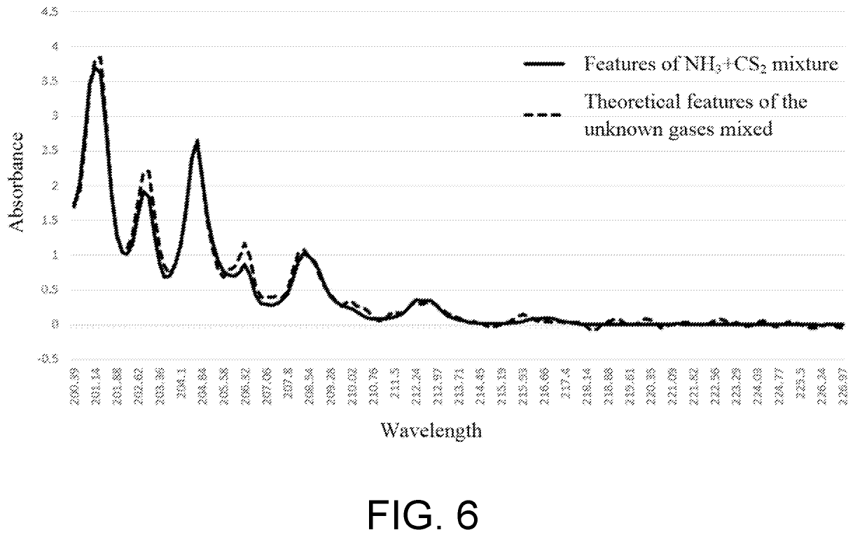

the correlation coefficient r is initialized as 0.3, and whether R is greater than 0.3 is determined; if yes, the unknown gas feature parameter Puⱼ is re-initialized, and S4 is performed; if not, S7 is performed;
S7, Kₘₐₓ=5000, ε=1e−3 is set, and when the iteration number K reaches 5000, the iteration is stopped and the final unknown gas feature D is calculated and obtained; the features of a mixture obtained after mixing NH₃ and CS₂ are obtained in this embodiment. A feature curve of the unknown gases obtained by use of this method is compared with an actual feature curve of the gases NH₃ and CS₂ mixed, which is shown in FIGS. 6; and
S8, the unknown gas feature obtained by iterative calculation is added to the known gas feature library, and according to the Lambert-Beer's law: d=C_{SO2}*D₁+C_{NO}*D₂+Cᵤ*Du, a formula of concentration inversion calculation according to the least square method is as follows:

$$( \begin{matrix}{c}_{So 2} \\ {c}_{N O} \\ {c}_{u}\end{matrix} ) = [ \begin{matrix}\begin{matrix}{D}_{1} * d & {D}_{2} * d\end{matrix} & Du * d\end{matrix} ] ( \begin{matrix}{D}_{1} * {D}_{1} & {D}_{2} * {D}_{1} & D u * {D}_{1} \\ {D}_{1} * {D}_{2} & {D}_{2} * {D}_{2} & D u * {D}_{2} \\ {D}_{1} * D u & {D}_{2} * D u & D u * D u\end{matrix} ) - 1.$$

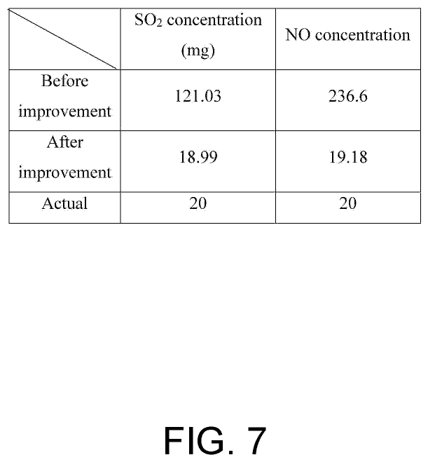

As shown in FIG. 7, in the Embodiment 2, calculated final concentrations of the gases SO₂ and NO are 18.99% and 19.18% respectively; under the condition that the features of the unknown gas are unavailable, concentrations of the gases SO₂ and NO obtained by direct inversion calculation are 121.03% and 231.6%, respectively; and actual concentrations of the gases SO₂ and NO are 20% and 20%, respectively.

According to the above two specific embodiments, it can be seen that the method provided by the present application enables to obtain features of one unknown gas or two or more mixed unknown gases. By adding the unknown gas feature to the known gas feature library, the concentration of the gas to be measured is obtained by inversion calculation. Through this method, the concentration of the gas to be measured is basically consistent with the actual data. The present application greatly improves the precision of gas concentration measurement, and eliminates the impact of an unknown interference gas on gas measurement precision to the greatest extent.

The present application aims to obtain the features of the unknown gas in a gas mixture, constructs a function model according to features of a known gas and features of the gas mixture, and finally obtains features of the unknown gas through learning and training. Features of any unknown gas different from the known gas can be obtained through this method, without limitation by any scene or gas type. The features of the unknown gas are incorporated into a feature library of the known gas, to provide more accurate parameters for obtaining the concentration of each component in the gas mixture through inversion calculation. The present application can effectively reduce cross interference caused by spectrum overlapping, eliminate the impact of an unknown interference gas on gas measurement precision to the greatest extent, and greatly improve the accuracy and precision of a gas measurement system.

The specific embodiments of the present application are mainly applied to the field of gas concentration measurement. This principle can also be used for recognition of unknown images, unknown solutions and waveforms, and applies to other fields without departing from the spirit of the present application. All these are considered to fall into the protection scope of the present application.

A person skilled in the art should understand that the embodiments of the present application may be provided in the form of a method, a system or a computer program product. Therefore, the present application may use a form of a complete hardware embodiment, a complete software embodiment or an embodiment combining software and hardware. Moreover, the present application may use a form of a computer program product that is implemented on one or more computer-usable storage media (including but not limited to a disk memory, a CD-ROM, an optical memory or the like) that include the computer-usable program code.

The present application is described with reference to the flowcharts and/or block diagrams of the method, the apparatus (systems), and the computer program product according to the embodiments of the present application. It should be understood that computer program instructions may be used to implement each procedure and/or each block in the flowcharts and/or the block diagrams and a combination of a procedure and/or a block in the flowcharts and/or the block diagrams. These computer program instructions may be provided to a general-purpose computer, a dedicated computer, an embedded processor or a processor of any other programmable data processing apparatus to generate a machine, so that the instructions executed by a computer or a processor of any other programmable data processing apparatus generate a device for implementing a specific function in one or more procedures in the flowcharts and/or in one or more blocks in the block diagrams.

These computer program instructions may also be stored in a computer-readable memory that can instruct the computer or any other programmable data processing apparatus to work in a specific manner, so that the instructions stored in the computer-readable memory generate manufactured products including an instruction device. The instruction device implements a specific function in one or more procedures in the flowcharts and/or in one or more blocks in the block diagrams.

These computer program instructions may also be loaded onto a computer or any other programmable data processing apparatus, so that a series of operations and steps are performed on the computer or any other programmable apparatus, so as to generate computer-implemented processing. Therefore, the instructions executed on the computer or any other programmable apparatus provide steps for implementing a specific function in one or more processes in the flowcharts and/or in one or more blocks in the block diagrams.

The present application provides a method and a system for measuring a concentration of a gas to be measured by identifying features of an unknown gas, to obtain the features of the unknown gas in a gas mixture, constructs a function model according to features of a known gas and features of the gas mixture, and finally obtains features of the unknown gas through learning and training. Features of any unknown gas different from the known gas can be obtained through this method, without limitation by any scene or gas type. The features of the unknown gas are incorporated into a feature library of the known gas, to provide more accurate parameters for obtaining the concentration of each component in the gas mixture through inversion calculation. The present application can effectively reduce cross interference caused by spectrum overlapping, eliminate the impact of an unknown interference gas on gas measurement precision to the greatest extent, and greatly improve the accuracy and precision of a gas measurement system.

What is claimed is:

1. A method for measuring a concentration of a gas to be measured by identifying features of an unknown gas, comprising the following steps:
S1, injecting standard samples of m known gases into a measuring cell of a gas analyzer, scanning in a wide spectral range to obtain an absorption spectrum of each sample, further obtaining a feature Dᵢ of each sample gas, and establishing a feature library D of the known gases;
S2, injecting a gas mixture into the measurement cell of the gas analyzer, and scanning in a wide spectral range to obtain the feature d of the gas mixture, where the gas mixture includes m known gases and n unknown gases, and features of the unknown gases are defined as Duⱼ;
S3, defining and initializing a known gas weight parameter w and an unknown gas weight parameter wu, and constructing a squared loss function and an objective function according to the known gas feature Dᵢ, the known gas weight parameter w, the unknown gas feature Duⱼ, the unknown gas weight parameter wu, and the gas mixture feature d;
S4, learning and training each parameter in the squared loss function until the value of the objective function is less than the set value or the number of learning times reaches the target number, and obtaining the final unknown gas feature Duⱼ; and
S5, adding the final unknown gas feature Duⱼ to the known gas feature library D, and obtaining the concentration of a gas to be measured in the gas mixture through gas concentration inversion calculation.

2. The method for measuring a concentration of a gas to be measured by identifying features of an unknown gas according to claim 1, wherein the squared loss function described in S3 is defined as follows:
$$θ = {( {\sum }_{i = 1}^{m} {w}_{i} * {D}_{i} + {\sum }_{j = 1}^{n} w {u}_{j} * D {u}_{j} - d )}^{2} ,$$
and the objective function min(θ) is constructed according to the squared loss function.

3. The method for measuring a concentration of a gas to be measured by identifying features of an unknown gas according to claim 2, wherein the unknown gas feature Duⱼ in S2 is defined as follows:
a gas feature function base Φ and a gas feature function space are established, the unknown gas parameter Puⱼ is defined and initialized, and the unknown gas feature Duⱼ is constructed according to the gas feature function base Φ and the unknown gas parameter Puⱼ.

4. The method for measuring a concentration of a gas to be measured by identifying features of an unknown gas according to claim 3, wherein an expression of constructing the unknown gas feature Duⱼ according to the gas feature function base Φ and the unknown gas parameter Puⱼ is as follows: Duⱼ=Puⱼ*Φ.

5. The method for measuring a concentration of a gas to be measured by identifying features of an unknown gas according to claim 4, wherein the process of learning and training each parameter in the squared loss function until the value of the objective function is less than the set value or the number of learning times reaches the target number, and obtaining the final unknown gas feature Duⱼ described in S4, comprises the following steps:
S41, using an iterative algorithm to learn and train the weight parameters w and wu and the unknown gas feature parameter Puⱼ, and recording an iteration number K;
S42, determining whether the remainder of K divided by a given integer N is zero, performing S43 if yes, and performing S44 if not;
S43, constructing a function of a correlation Rⱼ between the unknown gas feature Duⱼ and the known gas feature library D, initializing a correlation coefficient r, and determining whether Rⱼ is greater than r; if yes, re-initializing the unknown gas feature parameter Puⱼ, and performing S41; if not, performing S44; and
S44, determining whether the iteration number K reaches the set maximum K_max or whether the value of the objective function min(θ) is less than the set value ε; when at least any one of the above conditions is determined to be “yes”, stopping the iteration and calculating the final unknown gas feature Duⱼ; and when any of the above conditions is determined to be “not”, performing S41 and continuing learning and training.

6. The method for measuring a concentration of a gas to be measured by identifying features of an unknown gas according to claim 5, wherein in S41, the iterative algorithm is a gradient projection method with momentum.

7. The method for measuring a concentration of a gas to be measured by identifying features of an unknown gas according to claim 6, wherein in S43, the function of the correlation Rⱼ between the unknown gas feature Duⱼ and the known gas feature library D is as follows:
$$res = ( D {u}_{j} * D ) * {( D * D )}^{- 1} * D - D {u}_{j} ,$$
$${R}_{j} = \frac{1 - res * res}{D {u}_{j} * D {u}_{j}} .$$

8. The method for measuring a concentration of a gas to be measured by identifying features of an unknown gas according to claim 7, wherein in S5, the final unknown gas feature Duⱼ is added to the known gas feature library D, and the concentration of the gas to be measured in the gas mixture is obtained by inversion calculation according to the least square method.

9. A system for measuring a concentration of a gas to be measured by identifying features of an unknown gas, adopts the method for measuring a concentration of a gas to be measured by identifying features of an unknown gas according to claim 1, which comprises a light source, a measuring cell, a spectrometer, a storage module, a learning and training module, an initialization module, and an inversion module, wherein the light source is sequentially connected to the measurement cell and the spectrometer, the gas to be measured is injected into the measurement cell, and light emitted by the light source passes through the measurement cell filled with the gas to be measured, is received by the spectrometer and converted into a digital signal and then inputted to the storage module and the learning and training module respectively; the storage module is configured to store feature data of a variety of known gases; the learning and training module is configured to construct a squared loss function model between unknown gas features and known gas features and perform learning and training; the initialization module is connected to the learning and training module, and is configured to initialize the parameter data in the function model; the final unknown gas feature data obtained by learning and training is inputted into the storage module; the inversion module is connected with the storage module and configured to calculate the concentration of the gas to be measured, and the known gas feature data in the storage module and the final unknown gas feature data acquired by calculation are inputted into the inversion module for inversion calculation.

10. A system for measuring a concentration of a gas to be measured by identifying features of an unknown gas, adopts the method for measuring a concentration of a gas to be measured by identifying features of an unknown gas according to claim 2, which comprises a light source, a measuring cell, a spectrometer, a storage module, a learning and training module, an initialization module, and an inversion module, wherein the light source is sequentially connected to the measurement cell and the spectrometer, the gas to be measured is injected into the measurement cell, and light emitted by the light source passes through the measurement cell filled with the gas to be measured, is received by the spectrometer and converted into a digital signal and then inputted to the storage module and the learning and training module respectively; the storage module is configured to store feature data of a variety of known gases; the learning and training module is configured to construct a squared loss function model between unknown gas features and known gas features and perform learning and training; the initialization module is connected to the learning and training module, and is configured to initialize the parameter data in the function model; the final unknown gas feature data obtained by learning and training is inputted into the storage module; the inversion module is connected with the storage module and configured to calculate the concentration of the gas to be measured, and the known gas feature data in the storage module and the final unknown gas feature data acquired by calculation are inputted into the inversion module for inversion calculation.

11. A system for measuring a concentration of a gas to be measured by identifying features of an unknown gas, adopts the method for measuring a concentration of a gas to be measured by identifying features of an unknown gas according to claim 3, which comprises a light source, a measuring cell, a spectrometer, a storage module, a learning and training module, an initialization module, and an inversion module, wherein the light source is sequentially connected to the measurement cell and the spectrometer, the gas to be measured is injected into the measurement cell, and light emitted by the light source passes through the measurement cell filled with the gas to be measured, is received by the spectrometer and converted into a digital signal and then inputted to the storage module and the learning and training module respectively; the storage module is configured to store feature data of a variety of known gases; the learning and training module is configured to construct a squared loss function model between unknown gas features and known gas features and perform learning and training; the initialization module is connected to the learning and training module, and is configured to initialize the parameter data in the function model; the final unknown gas feature data obtained by learning and training is inputted into the storage module; the inversion module is connected with the storage module and configured to calculate the concentration of the gas to be measured, and the known gas feature data in the storage module and the final unknown gas feature data acquired by calculation are inputted into the inversion module for inversion calculation.

12. A system for measuring a concentration of a gas to be measured by identifying features of an unknown gas, adopts the method for measuring a concentration of a gas to be measured by identifying features of an unknown gas according to claim 4, which comprises a light source, a measuring cell, a spectrometer, a storage module, a learning and training module, an initialization module, and an inversion module, wherein the light source is sequentially connected to the measurement cell and the spectrometer, the gas to be measured is injected into the measurement cell, and light emitted by the light source passes through the measurement cell filled with the gas to be measured, is received by the spectrometer and converted into a digital signal and then inputted to the storage module and the learning and training module respectively; the storage module is configured to store feature data of a variety of known gases; the learning and training module is configured to construct a squared loss function model between unknown gas features and known gas features and perform learning and training; the initialization module is connected to the learning and training module, and is configured to initialize the parameter data in the function model; the final unknown gas feature data obtained by learning and training is inputted into the storage module; the inversion module is connected with the storage module and configured to calculate the concentration of the gas to be measured, and the known gas feature data in the storage module and the final unknown gas feature data acquired by calculation are inputted into the inversion module for inversion calculation.

13. A system for measuring a concentration of a gas to be measured by identifying features of an unknown gas, adopts the method for measuring a concentration of a gas to be measured by identifying features of an unknown gas according to claim 5, which comprises a light source, a measuring cell, a spectrometer, a storage module, a learning and training module, an initialization module, and an inversion module, wherein the light source is sequentially connected to the measurement cell and the spectrometer, the gas to be measured is injected into the measurement cell, and light emitted by the light source passes through the measurement cell filled with the gas to be measured, is received by the spectrometer and converted into a digital signal and then inputted to the storage module and the learning and training module respectively; the storage module is configured to store feature data of a variety of known gases; the learning and training module is configured to construct a squared loss function model between unknown gas features and known gas features and perform learning and training; the initialization module is connected to the learning and training module, and is configured to initialize the parameter data in the function model; the final unknown gas feature data obtained by learning and training is inputted into the storage module; the inversion module is connected with the storage module and configured to calculate the concentration of the gas to be measured, and the known gas feature data in the storage module and the final unknown gas feature data acquired by calculation are inputted into the inversion module for inversion calculation.

14. A system for measuring a concentration of a gas to be measured by identifying features of an unknown gas, adopts the method for measuring a concentration of a gas to be measured by identifying features of an unknown gas according to claim 6, which comprises a light source, a measuring cell, a spectrometer, a storage module, a learning and training module, an initialization module, and an inversion module, wherein the light source is sequentially connected to the measurement cell and the spectrometer, the gas to be measured is injected into the measurement cell, and light emitted by the light source passes through the measurement cell filled with the gas to be measured, is received by the spectrometer and converted into a digital signal and then inputted to the storage module and the learning and training module respectively; the storage module is configured to store feature data of a variety of known gases; the learning and training module is configured to construct a squared loss function model between unknown gas features and known gas features and perform learning and training; the initialization module is connected to the learning and training module, and is configured to initialize the parameter data in the function model; the final unknown gas feature data obtained by learning and training is inputted into the storage module; the inversion module is connected with the storage module and configured to calculate the concentration of the gas to be measured, and the known gas feature data in the storage module and the final unknown gas feature data acquired by calculation are inputted into the inversion module for inversion calculation.

15. A system for measuring a concentration of a gas to be measured by identifying features of an unknown gas, adopts the method for measuring a concentration of a gas to be measured by identifying features of an unknown gas according to claim 7, which comprises a light source, a measuring cell, a spectrometer, a storage module, a learning and training module, an initialization module, and an inversion module, wherein the light source is sequentially connected to the measurement cell and the spectrometer, the gas to be measured is injected into the measurement cell, and light emitted by the light source passes through the measurement cell filled with the gas to be measured, is received by the spectrometer and converted into a digital signal and then inputted to the storage module and the learning and training module respectively; the storage module is configured to store feature data of a variety of known gases; the learning and training module is configured to construct a squared loss function model between unknown gas features and known gas features and perform learning and training; the initialization module is connected to the learning and training module, and is configured to initialize the parameter data in the function model; the final unknown gas feature data obtained by learning and training is inputted into the storage module; the inversion module is connected with the storage module and configured to calculate the concentration of the gas to be measured, and the known gas feature data in the storage module and the final unknown gas feature data acquired by calculation are inputted into the inversion module for inversion calculation.

16. A system for measuring a concentration of a gas to be measured by identifying features of an unknown gas, adopts the method for measuring a concentration of a gas to be measured by identifying features of an unknown gas according to claim 8, which comprises a light source, a measuring cell, a spectrometer, a storage module, a learning and training module, an initialization module, and an inversion module, wherein the light source is sequentially connected to the measurement cell and the spectrometer, the gas to be measured is injected into the measurement cell, and light emitted by the light source passes through the measurement cell filled with the gas to be measured, is received by the spectrometer and converted into a digital signal and then inputted to the storage module and the learning and training module respectively; the storage module is configured to store feature data of a variety of known gases; the learning and training module is configured to construct a squared loss function model between unknown gas features and known gas features and perform learning and training; the initialization module is connected to the learning and training module, and is configured to initialize the parameter data in the function model; the final unknown gas feature data obtained by learning and training is inputted into the storage module; the inversion module is connected with the storage module and configured to calculate the concentration of the gas to be measured, and the known gas feature data in the storage module and the final unknown gas feature data acquired by calculation are inputted into the inversion module for inversion calculation.
